(19) United States
(12) Patent Application Publication
(10) Pub. No.: US 2022/0089787 A1
(43) Pub. Date: Mar. 24, 2022

(54) METHOD FOR MANUFACTURING PROTEOGLYCAN-CONTAINING COMPOSITION, AND PROTEOGLYCAN-CONTAINING COMPOSITION

(71) Applicant: LINISE CO., LTD., Sapporo-shi, Hokkaido (JP)

(72) Inventors: Hideharu NAKANO, Sapporo-shi, Hokkaido (JP); Masaki NARUMI, Date-shi, Hokkaido (JP)

(73) Assignee: LINISE CO., LTD., Sapporo-shi, Hokkaido (JP)

(21) Appl. No.: 17/423,197

(22) PCT Filed: Nov. 15, 2019
(86) PCT No.: PCT/JP2019/044919

§ 371 Date: Jul. 15, 2021

(30) Foreign Application Priority Data
Jan. 17, 2019 (JP) 2019-006386

(51) Int. Cl. C08B 37/00

(52) U.S. Cl. CPC C08B 37/0066

(57) ABSTRACT

A method for manufacturing a proteoglycan-containing composition including: a freezing step for freezing a starting material, where raw cartilage derived from fish is used as the starting material; and a freeze-drying step for freeze-drying frozen articles obtained in the freezing step. The method may also include an extraction step for furthermore adding an aqueous solvent to the resultant freeze-dried articles to carry out extraction. Additionally, a method for manufacturing a proteoglycan-containing composition including a mincing step for forming a starting material into surimi, where raw cartilage derived from fish is used as the starting material; and an extraction step for adding an aqueous solvent to the surimi obtained in the mincing step to carry out extraction.

### TECHNICAL FIELD

The present invention relates to a proteoglycan-containing composition, and more specifically relates to a proteoglycan-containing composition obtained from cartilage derived from fish.

### BACKGROUND ART

Broadly speaking, proteoglycans are a type of glycoprotein in which chondroitin sulfate, dermatan sulfate, heparan sulfate, heparin, keratan sulfate, or another sulfated polysaccharide known as a glycosaminoglycan can covalently bond to a core protein that forms a core structure. Proteoglycans are present in the extracellular matrices and cell surfaces of animals, and form complexes with hyaluronic acid or fibrous matrix proteins such as collagen.

A variety of functions have been reported in proteoglycans. For example, Patent Document 1 indicates that proteoglycans exhibit a TNF-α production-inhibiting action, an IFN-γ production-inhibiting action, an IL-10 production-promoting action, etc. Patent Document 2 indicates that proteoglycans have an action for promoting proliferation of skin fibroblasts. Patent Document 3 indicates that proteoglycans promote production of FGF-7 in hair papilla cells, and exhibit stimulatory action in said cells.

There are also various reports pertaining to methods for preparing proteoglycans. For example, Patent Document 4 discloses a preparation method in which a degreased dried powder obtained by implementing pulverization and degreasing treatments on salmon nasal cartilage is extracted using an extraction solvent, a crude proteoglycan is separated and refined from the resultant extract liquid, and dialysis is then performed. Patent Document 5 discloses a preparation method in which acetic acid is used as a solvent for eluting crude proteoglycans, the resultant eluate is filtered and then centrifuged, table-salt-saturated ethanol is added to a supernatant liquid, and the combination is again centrifuged to concentrate proteoglycans. Patent Document 6 discloses a method for preparing proteoglycans, the method including a step for immersing proteoglycan-containing animal tissue in a solution that contains at least peracetic acid, and a step for recovering the solution after said immersion. Patent Document 7 discloses a proteoglycan-containing substance that includes an acidic sugar component having a molecular weight of 2000 kDa or higher, and indicates that the proteoglycan-containing substance is prepared through aqueous extraction from fish cartilage that has been degreased through an ethanol treatment.

### RELATED ART DOCUMENTS

### Patent Documents

[Patent Document 1] Japanese Laid-Open Patent Application No. 2007-131548
[Patent Document 2] Japanese Laid-Open Patent Application No. 2008-247803 [Patent Document 3] Japanese Laid-Open Patent Application No. 2016-204297
[Patent Document 4] Japanese Laid-Open Patent Application No. 2001-172296
[Patent Document 5] Japanese Laid-Open Patent Application No. 2002-69097
[Patent Document 6] Japanese Laid-Open Patent Application No. 2012-201614
[Patent Document 7] International Publication No. 2011/007885

### DISCLOSURE OF THE INVENTION

### Problems the Invention is Intended to Solve

However, conventional methods for preparing proteoglycans have to a certain extent required, inter alia, steps in which degreasing is conducted through use of organic solvents, immersion in water, cleansing with ethanol, etc. However, such methods have disadvantageous aspects in that the implementation thereof takes time and the form in which the proteoglycans are naturally present is inevitably destroyed as the steps are passed through.

It is an object of the present invention to provide: a method for manufacturing a proteoglycan-containing composition, the method being configured so that proteoglycans are obtained while remaining in essentially their natural form; and a proteoglycan-containing composition obtained through said method.

### Means for Solving the Aforementioned Problems

As a result of thorough investigations for the purpose of achieving the abovementioned object, the inventors discovered that when raw cartilage derived from fish is used as a starting material and is extracted without being subjected to freezing and thawing processes, contamination by lipids from the starting material is minimized and proteoglycans are obtained at high yield while remaining in essentially their natural form, whereupon the inventors perfected the present invention.

Specifically, according to a first aspect, the present invention provides a method for manufacturing a proteoglycan-containing composition, characterized in comprising: a freezing step for freezing a starting material, where raw cartilage derived from fish is used as the starting material; and a freeze-drying step for freeze-drying frozen articles obtained in the freezing step.

According to the manufacturing method in the first aspect, because raw cartilage derived from fish is used as the starting material, and because the starting material is frozen and then immediately freeze-dried without resultant frozen articles being allowed to thaw, denaturation and degeneration of the starting material are minimized, the proteoglycans are consequently included in essentially their natural form, and it is possible to provide an ingredient in which the proteoglycans can be used.

In the manufacturing method according to the first aspect, in the freezing step, the starting material is preferably frozen so as to reach a temperature band ranging from at least −5° C. to less than 0° C. over 30 minutes or more. Accordingly, the starting material reaches the temperature band ranging from at least −5° C. to less than 0° C., this being the temperature band in which ice crystals are generated (ice crystal generation temperature band), whereby ice crystals in the starting material adequately grow and increase in size. This makes it possible for cartilage tissues to be more adequately broken down and, consequently, to form an ingredient in which proteoglycans in essentially their natural form can be even more readily used.

In the manufacturing method according to the first aspect, the method preferably includes an extraction step for furthermore adding an aqueous solvent to freeze-dried articles obtained in the freeze-drying step to carry out extraction. Accordingly, because the raw cartilage derived from fish is used as a starting material and is extracted without being subjected to freezing and thawing processes, denaturation and degeneration of the starting material are minimized, contamination by lipids from the starting material is consequently minimized, and there is obtained a proteoglycan-containing composition that contains proteoglycans in essentially their natural state. Additionally, it is possible to carry out efficient extraction without using an organic solvent, and no problems are presented in terms of safety, such as for oral ingestion by humans or application to the skin.

According to a second aspect, the present invention provides a method for manufacturing a proteoglycan-containing composition, characterized in comprising: a mincing step for forming a starting material into surimi, where raw cartilage derived from fish is used as the starting material; and an extraction step for adding an aqueous solvent to the surimi obtained in the mincing step to carry out extraction.

According to the manufacturing method in the second aspect, because raw cartilage derived from fish is used as the starting material and is extracted without being subjected to freezing and thawing processes, denaturation and degeneration of the starting material are minimized, contamination by lipids from the starting material is consequently minimized, and there is obtained a proteoglycan-containing composition that contains proteoglycans in essentially their natural state. Additionally, it is possible to carry out efficient extraction without using an organic solvent, and no problems are presented in terms of safety, such as for oral ingestion by humans or application to the skin.

In the manufacturing method according to the first and second aspects, the method preferably includes a drying step for furthermore drying extracted articles obtained in the extraction step. Accordingly, decomposition, etc., is prevented, and storage properties are improved.

In the manufacturing method according to the first and second aspects, dried articles obtained in the drying step preferably contain 36 mass % or more of proteoglycans and contain 36 mass % or more of collagen.

According to a third aspect, the present invention provides a proteoglycan-containing composition formed from a cartilage extract derived from fish, the composition containing 36 mass % or more of proteoglycans and 36 mass % or more of collagen, and the mass ratio of the proteoglycans and the collagen being 1:1.7 to 1.25:1.

In the composition described above, the weight-average molecular weight of the proteoglycans is preferably 2,000,000-4,150,000 Daltons.

In the composition described above, the lipid content is preferably 1 mass % or less.

In the composition described above, proteoglycans having a weight-average molecular weight within the range of 2,000,000-3,400,000 Daltons preferably occupy 30 mass % or more of the composition.

### Effect of the Invention

The present invention provides a high-quality proteoglycan ingredient that can be applied to cosmetic products, functional food products, pharmaceutical products, etc.

### BRIEF DESCRIPTION OF THE DRAWINGS

FIG. 1 is a step diagram showing one embodiment of the method for manufacturing a proteoglycan-containing composition according to the present invention;

FIG. 2 is a step diagram showing another embodiment of the method for manufacturing a proteoglycan-containing composition according to the present invention;

FIG. 3 is a step diagram showing yet another embodiment of the method for manufacturing a proteoglycan-containing composition according to the present invention;

FIG. 4 is a step diagram showing still another embodiment of the method for manufacturing a proteoglycan-containing composition according to the present invention;

FIG. 5 is an example of various HPLC plots of the results of HPLC analyses conducted in experiment example 1, when pulverulent compositions obtained in examples 1 to 3 and comparative example 1 were analyzed;

FIG. 6 is a set of photographs showing results of investigating the effect of freezing conditions in experiment example 2, where FIG. 6(a) is a microphotograph taken when a cross-section of a raw article of a fragment of salmon head cartilage (cartilage from the head) is stained using Alcian blue (blue stain), FIG. 6(b) is a microphotograph taken when a cross-section of a flash-frozen article is stained using Alcian blue (blue stain), and FIG. 6(c) is a microphotograph taken when a cross-section of an article that has been frozen under slow-freezing conditions in the same manner as during preparation of the pulverulent compositions in examples 1 and 3 is stained using Alcian blue (blue stain);

FIG. 7 is an explanatory diagram in which the state where proteoglycans are present in a living body is schematically represented; and

FIG. 8 is a set of explanatory diagrams schematically showing the structure of a proteoglycan ingredient obtained through the method for manufacturing a proteoglycan-containing composition according to the present invention, where FIG. 8(a) is an explanatory drawing schematically showing a state where proteoglycans are present in a dimer, FIG. 8(b) is an explanatory drawing schematically showing a state where proteoglycans are present in a trimer, and FIG. 8(c) is an explanatory drawing schematically showing a state where proteoglycans are present in a tetramer.

### BEST MODE FOR CARRYING OUT THE INVENTION

In the present invention, raw cartilage derived from fish is used as a basic source of proteoglycans. There are no particular limitations as to the type of fish, the site of cartilage tissue therein, etc.; examples include salmon nasal cartilage (head cartilage), shark cartilage, ray cartilage, and squid cartilage. Salmon nasal cartilage (head cartilage) in particular is more desirable due to not only having a high proteoglycan content but also being cheaply available as a site that is generally discarded in the field of fishery processing. For example, in processing of roe or fillets of salmon, the heads from landed salmon are discarded and disposed of in large quantities, and therefore it is possible to acquire the heads and to harvest and use the nasal cartilage from the heads.

In the present description, “raw cartilage” refers to a starting material that has not had a history of reaching temperatures of 30° C. or higher and also has not undergone freezing or thawing processes. As indicated in the examples (described below), freezing and thawing result in denaturation and degradation of the proteoglycans and make it difficult to extract the proteoglycans in essentially their natural form. Generally, with starting materials that have had a history of reaching temperatures of 30° C. or higher, denaturation and degradation readily occur in biomolecules such as proteins, and extraction of proteoglycans in essentially their natural form is more difficult; therefore, such starting materials are not preferred. In order to avoid, inter alia, propagation of microorganisms, it is preferable to prepare the cartilage immediately after arrival from an affiliated fishery processor, etc., without leaving a period of time from landing of the fish.

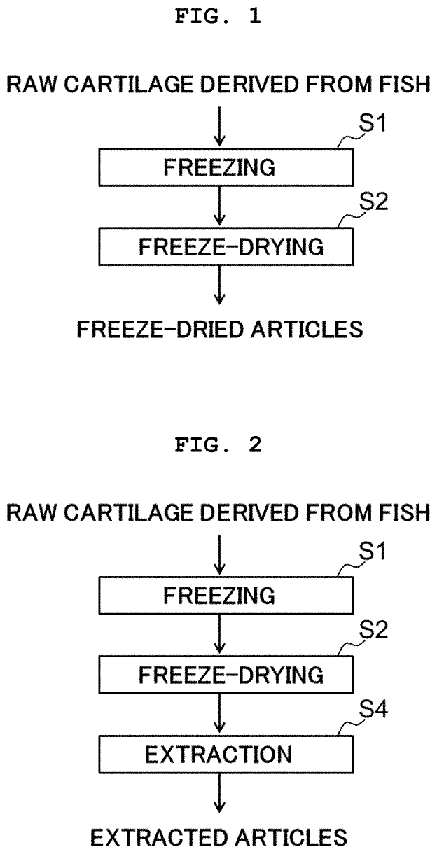

FIG. 1 shows one embodiment of the method for manufacturing a proteoglycan-containing composition according to the present invention. In this embodiment, the proteoglycan-containing composition is obtained through: a step (indicated by S1 in FIG. 1) for freezing a starting material, where raw cartilage derived from fish is used as the starting material; and a step (indicated by S2 in FIG. 1) for freeze-drying frozen articles obtained by freezing. The freezing can be conducted by means such as using a freezing device that is generally well known to persons skilled in the art or allowing the starting material to stand inside a freezing chamber. There is no particular limitation as to the temperature conditions for freezing; it is suitable to, inter alia, cool the starting material until a temperature ranging from −40° C. to −10° C. is reached, and it is more preferable to cool the starting material until a temperature ranging from −40° C. to −30° C. is reached in order to achieve more complete freezing and to avoid the occurrence of even partial or temporary freezing and thawing. The drying can be conducted by means such as a vacuum freeze dryer that is generally well known to persons skilled in the art. Generally, the setting conditions for the freeze-drying include a shelf temperature ranging from −40° C. to 50° C., etc., and an in-chamber vacuum of 0.1 to 2000 Pa, etc.

As a more preferred aspect of the freezing step in the present invention, it is preferable for the freezing of the starting material in the freezing step to be conducted under slow-freezing conditions. “Slow-freezing” refers to a process for freezing the starting material so that the starting material freezes over a prescribed time within a temperature band ranging from −5° C. to less than 0° C., this being the ice crystal generation temperature band, whereby ice crystals in the starting material are adequately grown and enlarged. This makes it possible for cartilage tissues to be more adequately broken down and, consequently, to form an ingredient in which proteoglycans in essentially their natural form can be even more readily used. Specifically, slow-freezing involves, inter alia, using a freezing device in which temperature transition conditions are set or allowing the starting material to stand inside a freezing chamber that has been set to suitable temperature conditions. For example, slow-freezing can be performed by, inter alia, freezing the starting material so that the starting material reaches the temperature band ranging from at least −5° C. to less than 0° C. over 30 minutes or more.

Post-freeze-drying dried articles prepared in this manner include proteoglycans in a readily usable state. Specifically, the proteoglycans are included in a state where, for example, water or other aqueous solvents may elute easily thereof. The proteoglycans are also included in a state where, by being orally administered to humans or applied to the skin, the proteoglycans easily come into contact with a living body and are consequently used by the living body. Furthermore, because moisture is removed, decomposition, etc., is prevented, and exceptional storage stability is also achieved. Thus, such dried articles have extremely high value as an ingredient for supplying proteoglycans.

The abovementioned post-freeze-drying dried articles may be pulverized by means such as pulverizers, mills, and mass corroders that are generally well known to persons skilled in the art. This yields a form that is more easily used as an ingredient for supplying proteoglycans. As pertains to the granularity of the post-pulverization pulverized articles, it is preferable to pulverize the articles to an extent such that about 90 mass % or more of the entirety passes through a 30-mesh sieve (mesh size: 500 μm), and more preferable to pulverize the articles to an extent such that about 90 mass % or more of the entirety passes through a 60-mesh sieve (mesh size: 250 μm). Alternatively, it is preferable to prepare the pulverized articles so that about 90 mass % or more of the entirety passes screening at a diameter of 0.3 mm or more to 0.75 mm or less.

FIG. 2 shows another embodiment of the method for manufacturing a proteoglycan-containing composition according to the present invention. In this embodiment, the proteoglycan-containing composition is obtained through: a step (indicated by S1 in FIG. 2) for freezing a starting material, where raw cartilage derived from fish is used as the starting material; a step (indicated by S2 in FIG. 2) for freeze-drying frozen articles obtained by freezing; and a step (indicated by S4 in FIG. 2) for adding an aqueous solvent to the freeze-dried articles obtained by freeze-drying to carry out extraction. The means, etc., for freezing and freeze-drying are as described above. There is no particular limitation as to the aqueous solvent with which extraction is performed, provided that the solvent is water or has water as a main component; however, it is preferable to use an aqueous solvent that has been adjusted so as to be more alkaline using an alkaline agent, such as sodium hydroxide, calcium hydroxide, potassium hydroxide, calcium carbonate, sodium bicarbonate, or ammonium carbonate. The pH of the aqueous solvent is preferably about 7 to 12, more preferably about 8 to 12, and even more preferably about 9 to 12. Sodium hydroxide is preferred as the alkaline agent. As pertains to extraction conditions, it is possible to add the aqueous solvent in an amount of 100 to 140 times that of the pulverized freeze-dried articles, and to carry out extraction by, inter alia, processing the mixture at 10.5 to 14.5° C. for 0.5 to 10 hours. In this case, the extraction process may be carried out by having the aqueous solvent to which the pulverized freeze-dried articles were added be introduced into a suitable container and allowing the combination to stand, and may be carried out while shaking the container or stirring the articles using suitable stirring means in order to more efficiently carry out extraction. However, with excessively vigorous stirring, there is a risk that denaturation or degeneration of the proteoglycans in essentially their natural form will occur, and therefore such stirring is not preferred.

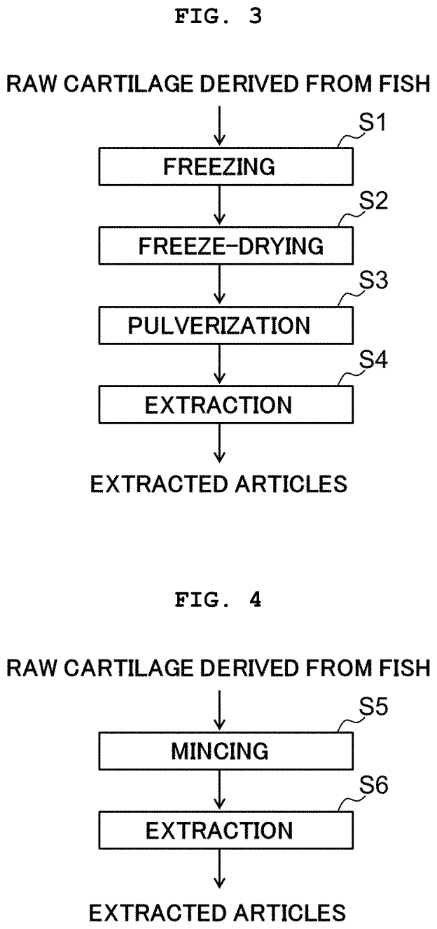

FIG. 3 shows yet another embodiment of the method for manufacturing a proteoglycan-containing composition according to the present invention. In this embodiment, the proteoglycan-containing composition is obtained through: a step (indicated by S1 in FIG. 3) for freezing a starting material, where raw cartilage derived from fish is used as the starting material; a step (indicated by S2 in FIG. 3) for freeze-drying frozen articles obtained by freezing; a step (indicated by S3 in FIG. 3) for pulverizing the freeze-dried articles obtained by freeze-drying; and a step (indicated by S4 in FIG. 3) for adding an aqueous solvent to the freeze-dried pulverized articles obtained in the pulverization step to carry out extraction. The means, etc., for freezing, freeze-drying, and extraction by aqueous solvent are as described above. The pulverization of the freeze-dried articles can be performed using means such as pulverizers, mills, and mass corroders that are generally well known to persons skilled in the art. As pertains to the granularity of the post-pulverization pulverized articles, it is preferable to pulverize the articles to an extent such that about 90 mass % or more of the entirety passes through a 30-mesh sieve (mesh size: 500 μm), and more preferable to pulverize the articles to an extent such that about 90 mass % or more of the entirety passes through a 60-mesh sieve (mesh size: 250 μm). Alternatively, it is preferable to prepare the pulverized articles so that about 90 mass % or more of the entirety passes screening at a diameter of 0.3 mm or more to 0.75 mm or less.

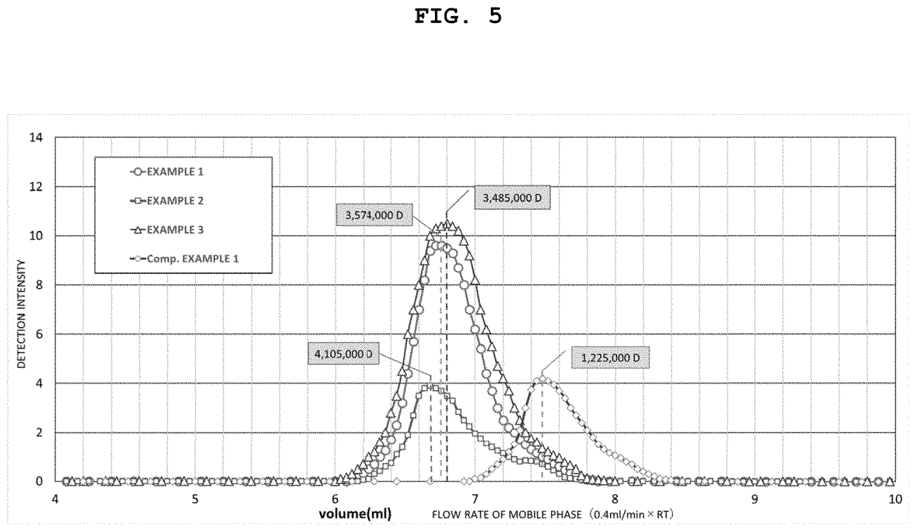

FIG. 4 shows still another embodiment of the method for manufacturing a proteoglycan-containing composition according to the present invention. In this embodiment, the proteoglycan-containing composition is obtained through: a step (indicated by S5 in FIG. 4) for mincing a starting material, where raw cartilage derived from fish is used as the starting material; and a step (indicated by S6 in FIG. 4) for adding an aqueous solvent to the minced surimi to carry out extraction. The mincing can be performed using means such as meat grinders, meat choppers, and homogenizers that are generally well known to persons skilled in the art. The means, etc., for extraction by aqueous solvent are as described above, except that the post-freeze-drying dried articles are replaced with surimi.

The extracted articles obtained through extraction by the aqueous solvent described above may be dried by means such as reduced-pressure dryers and atomizing dryers that are generally well known to persons skilled in the art, and the dried articles may furthermore be dried and powderized by grinding, pulverization, etc. If the articles are in a dried form, moisture is removed in the same manner as with the post-freeze-drying dried articles described above, therefore preventing decomposition, etc., and yielding exceptional storage stability. During drying, it may be pharmaceutically acceptable to add dextrin or another diluent, crystalline cellulose, silica, and the like.

For the purpose of powderization, it is possible to use means such as pulverizers, mills, and mass corroders that are generally well known to persons skilled in the art, in the same manner as with the freeze-dried articles of cartilage described above. As pertains to the granularity after powderization, it is preferable to powderize the articles to an extent such that about 90 mass % or more of the entirety passes through a 30-mesh sieve (mesh size: 500 μm), and more preferable to powderize the articles to an extent such that about 90 mass % or more of the entirety passes through a 60-mesh sieve (mesh size: 250 μm). Alternatively, it is preferable to powderize the articles so that about 90 mass % or more of the entirety passes screening at a diameter of 0.3 mm or more to 0.75 mm or less.

Preparing the composition in the manner described above makes it possible to obtain a proteoglycan ingredient that has a high proteoglycan content. The composition preferably contains 36 mass % or more of proteoglycans, and it is possible to obtain a proteoglycan-containing composition that contains 36 mass % or more of collagen. It is thought that the proteoglycans in the composition are present within a molecular weight range of 2,000,000 to 4,150,000 Daltons in terms of weight-average molecular weight, and moreover are present in the form of dimers, trimers, or tetramers. The mass ratio of the proteoglycans and the collagen is about 1:1.7 to 1.25:1. Specifically, a proteoglycan ingredient containing proteoglycans that are more essentially natural is formed. Contamination by lipids from the starting material is low, the lipid content of the composition preferably being 1 mass % or less.

Examples of the method for measuring the proteoglycan content include methods in which the amount of uronic acid, being a degradation product of proteoglycans, is measured by HPLC analysis or the Galambos method (carbazole sulfuric acid method), and the proteoglycan content is converted from the measured value. Examples of the method for measuring the collagen content include methods in which the hydroxypropyl content is measured by amino acid compositional analysis, and the collagen content is converted from the measured value. Examples of the method for measuring the lipid content include acidolysis methods, which are well known as methods for measuring the lipid content of food products.

As described below, examples of more accurately measuring the molecular weight of biopolymers include structural analysis conducted through static light scattering methods in which a multi-angle light scattering detector is used.

In addition to the proteoglycans and the collagen, it is permissible to furthermore add vitamin C, imidazole peptides, collagen peptides, salmon ovary peptides, β-hydroxy-β-methylbutyrate (HMB), etc., to the proteoglycan-containing composition obtained according to the present invention.

The proteoglycan-containing composition obtained according to the present invention can have uses for, e.g., cosmetic products, health food products and supplements, pharmaceutical products, and quasi-pharmaceutical products, and in particular can be suitably used as a starting material ingredient thereof. This composition also can have uses for pet animals and other animals, as well as humans.

### EXAMPLES

The present invention is specifically described using the examples below, but these examples do not in any way limit the scope of the present invention.

### Example 1

A salmon head discharged from a fishery processing facility was acquired and nasal cartilage was harvested from the head to collect about 28 g of raw cartilage from the salmon head. The raw cartilage was allowed to stand and frozen inside a freezing chamber set to a setting temperature of −30° C. to obtain a frozen article of salmon nasal cartilage. At this time, for freezing conditions, the freezing was conducted under slow-freezing conditions (rather than flash-freezing). Specifically, the starting material was frozen so that the product temperature thereof reached a temperature band ranging from at least −5° C. to less than 0° C., this being the ice crystal generation temperature band, over about 30 minutes. The resultant frozen article was freeze-dried using a vacuum freeze-drying device and furthermore was pulverized using a pin mill pulverizer (product name: “Sample mill (SAM),” produced by Nara Kikai Seisakusho KK) so that about 90 mass % or more of the entirety would pass screening at a diameter of 0.3 mm or more to 0.75 mm or less. The resultant freeze-dried pulverized articles were added to 120 mL of pure water (distilled water) so as to reach a final concentration of 10 w/v %, and the solution was shaken for 15 minutes at a temperature of 11° C. in each container. Solid-liquid separation was then performed through centrifugation, a liquid fraction was recovered, and drying was carried out using a vacuum drying device to obtain a milk-white pulverulent composition.

### Example 2

Salmon heads discharged from a fishery processing facility were acquired and nasal cartilage was harvested from the heads to collect about 1 kg of raw cartilage from 44 salmon heads. The raw cartilage was minced using a meat chopper device (setting temperature: 10° C.) to obtain surimi paste. The resultant surimi was added to 120 mL of pure water (distilled water) so as to reach a final concentration of 10 w/v % while care was taken so that the product temperature did not rise excessively, and the solution was shaken for 15 minutes at a temperature of 11° C. in each container. Solid-liquid separation was then performed through centrifugation, a liquid fraction was recovered, and drying was carried out using a vacuum drying device to obtain a pulverulent composition having a color tone ranging from milk-white to pale yellow.

### Example 3

A milk-white pulverulent composition was obtained in the same manner as in example 1, except that the solvent with which extraction from the freeze-dried pulverized articles was performed was a 0.005% NaOH aqueous solution (pH value: 11.1) rather than pure water (distilled water).

### Comparative Example 1

A pulverulent composition was prepared in the same manner as in example 2, except that the collected raw cartilage used was flash-frozen for a brief time using a freezing device (setting temperature: −25° C.), and then thawed under 10° C. flowing water. The color tone of the resultant pulverulent composition ranged from milk-white to pale yellow.

### Experiment Example 1

The pulverulent compositions obtained in examples 1 to 3 and comparative example 1 were investigated, in conformance with usual methods, with respect to the amount of proteoglycans contained in the compositions and the molecular weight of the compositions. The main analysis conditions employed for HPLC analysis were as follows.

(Analysis Conditions)
Size exclusion chromatography column: TSKgel G6000 PWXL (7.8 mm×300 mm), exclusion boundary: 50,000,000
Guard column: TSKgel guard column PWXL (6.0 mm×40 mm)
Column temperature: 40° C.
Mobile phase: 0.1M phosphate buffer in 0.1M NaCl (pH value: 7.0)
Flow rate: 0.4 mL/min
Detector: RI (differential refraction)
Proteoglycan preparation for quantitative analysis: Salmon Nasal Cartilage Proteoglycan (Cosmo Bio KK)
Pullulan preparation for molecular weight: STD P-800 Mw 80.5×10⁴, P-400 Mw 36.6×10⁴, P-200 Mw 20.0×10⁴ (Showa Denko KK)

In quantitative analysis of proteoglycans, the preparation indicated above was used, and both a method for producing and quantifying calibration curves on the basis of peak area or peak height from HPLC plots of a sample having a known concentration and a method for measuring the amount of uronic acid through the Galambos method (carbazole sulfuric acid method) to thereby carry out quantification were used in combination therefor.

Moreover, in analysis of molecular weight, the preparations having the three molecular weights indicated above were used, and calibration curves were created on the basis of retention time (holding time) at the peak positions of the HPLC plots.

The results are shown in table 1 and FIG. 5.

TABLE 1

| | | Yielding proportion | | |
|---|---|---|---|---|
| | | relative to raw | | |
| | | cartilage used as | | |
| | Amount of | starting material | | |
| | proteoglycans | (percentage in terms | Molecular | HPLC |
| | (concentration) | of mass) (%) | weight | plot |
| Example 1 | HPLC method: 43.2 | 6.5 (based on HPLC- | 3,574,000 | FIG. |
| | mass % | quantified value) | Daltons | 5 |
| | Galambos method: | | Composition | |
| | 41.2 mass % | | ratio: 100% | |
| Example 2 | HPLC method: 20.9 | 3.7 (based on HPLC- | 4,105,000 | |
| | mass % | quantified value) | Daltons | |
| | Galambos method: | | Composition | |
| | 20.5 mass % | | ratio: 100% | |
| Example 3 | HPLC method: 42.1 | 6.85 (based on HPLC- | 3,485,000 | |
| | mass % | quantified value) | Daltons | |
| | Galambos method: | | Composition | |
| | 41.5 mass % | | ratio: 100% | |
| Comparative | HPLC method: 21.2 | 3.8 (based on HPLC- | 1,225,000 | |
| example 1 | mass % | quantified value) | Daltons | |
| | Galambos method: | | Composition | |
| | 21.0 mass % | | ratio: 91.8% | |

In the results, in example 1, in which raw cartilage from salmon was used as the starting material and was freeze-dried before being extracted, a peak was exhibited at a position of 3,574,000 Daltons on the HPLC plot (FIG. 5). In example 2, in which raw cartilage from salmon was used as the starting material and was minced to obtain surimi paste before being extracted, a peak was exhibited at a position of 4,105,000 Daltons on the HPLC plot (FIG. 5). In example 3, a peak was exhibited at a position of 3,485,000 Daltons on the HPLC plot (FIG. 5). However, in comparative example 1, in which raw cartilage from salmon that had been flash-frozen for a brief time and then thawed was used as the starting material, a peak was exhibited at a position of 1,225,000 Daltons on the HPLC plot (FIG. 5), and the yield was worse than that in examples 1 and 3 and roughly equivalent to that in example 2. It is thus thought that, when proteoglycans are extracted without the starting material having been subjected to freezing and thawing processes, it is possible to extract proteoglycans that are more essentially natural, without degrading or denaturing the proteoglycans, to a greater extent than in cases where the starting material has been subjected to freezing and thawing processes.

### Experiment Example 2

The effects of freezing conditions were examined as follows. Specifically, slices of each of a raw article of a fragment of salmon head cartilage (cartilage from the head), an article flash-frozen to −20° C., and an article that had been frozen under slow-freezing conditions (freezing conducted so that the starting material reached a temperature band ranging from at least −5° C. to less than 0° C. over about 30 minutes) in the same manner as during preparation of the pulverulent compositions in examples 1 and 3 were produced, and the slices were stained using Alcian blue (blue stain), which is a proteoglycan staining reagent, and were observed using a microscope.

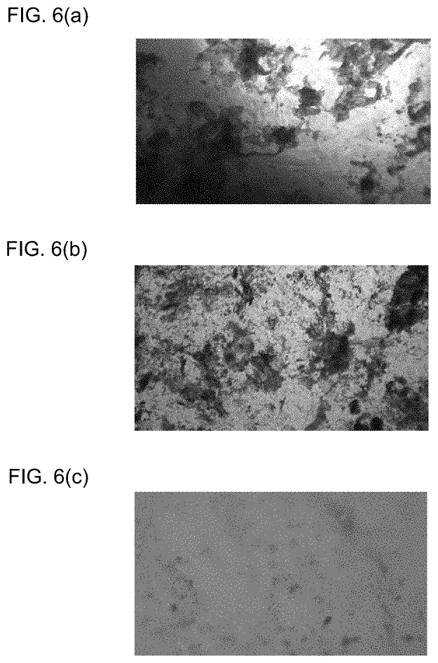

In the results, under flash-freezing conditions, the condition of the stain was similar to that from before freezing, and the proteoglycans remained in the tissue (FIG. 6b). However, in the case of freezing under slow-freezing conditions, the stain became fainter, and the proteoglycans were eluted from the tissue (FIG. 6c). It is thus thought that employing slow-freezing conditions when preparing the freeze-dried articles makes it possible to extract the proteoglycans in essentially their natural form at higher yield than in the case of flash-freezing.

### Experiment Example 3

Amino acid compositional analysis was conducted on the pulverulent composition obtained in example 1. The amino acid compositional analysis involved supplying a hydrolyzed product obtained after hydrolysis of a sample to an amino acid automated analysis device in conformance with usual methods to measure the amounts of amino acids. As a result, a conversion factor of 12.51 was used to calculate the amount of collagen in the obtained hydroxyproline content (mg/100 g), whereupon it was deemed that the collagen content was 41 mass %.

The lipid content of the pulverulent composition obtained in example 1 was 0.6 mass %, as measured by acidolysis in conformance with usual methods. The measurement of the lipid content using acidolysis was conducted by heating the sample using hydrochloric acid to conduct hydrolysis, subsequently using a Mojonnier tube to conduct extraction using diethyl ether and petroleum ether, and drying the resultant liquid extract and measuring the weight thereof.

The hyaluronic acid content of the pulverulent composition obtained in example 1, as estimated by treating the sample with actinomycete hyaluronidase and then detecting hyaluronic acid degradation products (low-molecular-weight sugars), was less than 1 mass %.

### Experiment Example 4

It is thought that the magnitude of error in experiment example 1 was high because a size exclusion chromatography column was used and the molecular weight was derived by comparison relative to the pullulan preparation. In addition, it was impossible to predict molecular shapes such as rigid spheres, rods, and random coils. Thus, there was implemented a structural analysis conducted through static light scattering methods in which a SEC-MALS method was used, these methods enabling more absolute measurement of molecular weight and making it possible to obtain information relating to molecular shape as well. “SEC” is an abbreviation of “size exclusion chromatograph,” and “MALS” is an abbreviation of “multi-angle light scattering” (multi-angle light scattering detector).

The pulverulent composition obtained in example 1 was analyzed as a sample, and two commercially available proteoglycan ingredient products were analyzed as controls. The analysis was conducted in conformance with usual methods. The main analysis conditions employed were as follows.

(Analysis Conditions)
Multi-angle light scattering detector: Dawn Heleos II (Wyatt Technology)
Size exclusion chromatography column: Shodex SB-807 (exclusion boundary: 50,000,000)
Detector 1: differential refractive index detector Optilab T-rEX (Wyatt Technology)
Detector 2: viscosity detector ViscoStar III (Wyatt Technology)
Mobile phase: 0.1M phosphoric acid buffer
Flow rate: 0.5 mL/min
Temperature: 40° C.
do/dc value: 0.16 mg/L (reference value)

The results are shown in table 2.

TABLE 2

| | Example 1 | Company A | Company B |
|---|---|---|---|
| Mw (g/mol) | 3.01 × 10⁶ | 1.17 × 10⁶ | 0.41 × 10⁶ |
| weight-average | | | |
| molecular weight | | | |
| Rz (nm) | 81.9 (±0.9%) | 48.4 (±1.7%) | 36.9 (±2.8%) |
| rotation radius | | | |
| RMS conformation | 0.53 | 0.26 | 0.1 |
| plot slope | | | |
| Molecular shape | Random | Rigid | Ultra-rigid |
| | coils | spheres | spheres |

In the results, whereas the weight-average molecular weight of the proteoglycans included in the pulverulent composition obtained in example 1 was 3,010,000 Daltons, the value for one of the commercially available proteoglycan ingredient products was 1,170,000 Daltons and the value for the other was 410,000 Daltons, both of the latter being lower molecular weights than that in example 1. In addition, as pertains to the values for the rotation radius and the slope in an RMS conformation plot, which are parameters relating to molecular shape, whereas the results indicated that the proteoglycans included in the pulverulent composition obtained in example 1 had a molecular shape of random coils, the results also indicated that the commercially available proteoglycan ingredient products had a molecular shape of rigid spheres or ultra-rigid spheres.

According to the results described above, it is thought that, whereas proteoglycans were obtained in essentially their natural state in example 1 because the pulverulent composition obtained in example 1 was prepared without undergoing freezing and thawing processes, the proteoglycans in the controls were denatured or degraded at least to a molecular weight of 1,500,000 Daltons or less during the preparation process due to the commercially available proteoglycan ingredient products not being prepared using such a method.

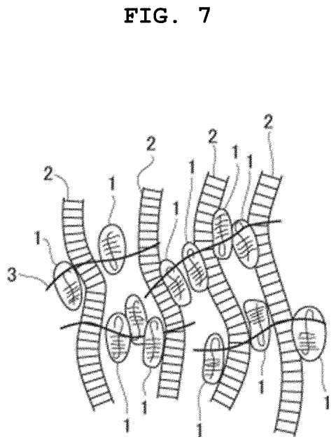

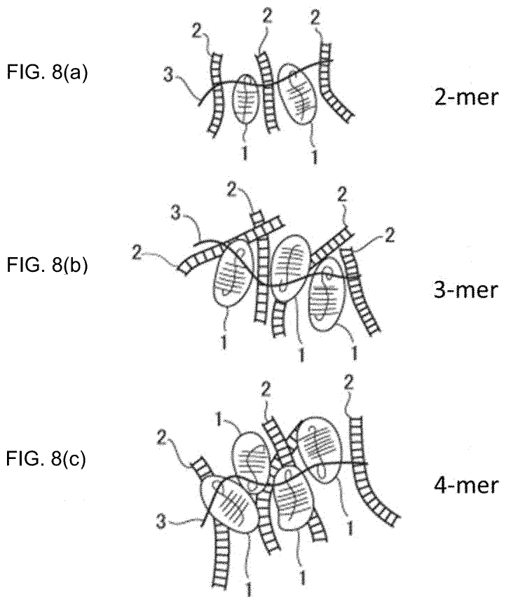

As schematically shown in FIG. 7, in living tissues, proteoglycans (indicated by reference 1 in FIG. 7) constitute an extracellular matrix together with collagen molecules (indicated by reference 2 in FIG. 7) and hyaluronic acid molecules (indicated by reference 3 in FIG. 7). Thus, it can be understood that a plurality of proteoglycans are connected via collagen or hyaluronic acid to form multimers. In this respect, when estimating from the molecular weight measured as described above, it is thought that dimers, trimers, and tetramers, among the natural multimeric forms, were obtained in example 1 (refer to FIG. 8). It is thought that the hyaluronic acid content, indicated by reference 3 in FIG. 8, is less than 1 mass % in the results of experiment example 3 and is not included in as large quantities as the proteoglycan content or the collagen content.

1-10. (canceled)

11. A method for manufacturing a proteoglycan-containing composition, characterized in comprising: a freezing step for freezing a starting material, where raw cartilage that has not had a history of reaching temperature of 30° C. or higher and has not undergone freezing derived from fish is used as the starting material; a freeze-drying step for freeze-drying frozen articles obtained in the freezing step; and an extraction step for adding an aqueous solvent to freeze-dried articles obtained in the freeze-drying step to carry out extraction.

12. The method for manufacturing a proteoglycan-containing composition according to claim 11, wherein, in the freezing step, the starting material is frozen so as to reach a temperature band ranging from at least −5° C. to less than 0° C. over 30 minutes or more.

13. The method for manufacturing a proteoglycan-containing composition according to claim 11, wherein the method includes a drying step for furthermore drying extracted articles obtained in the extraction step.

14. The method for manufacturing a proteoglycan-containing composition according to claim 13, wherein dried articles obtained in the drying step contain 36 mass % or more of proteoglycans and contain 36 mass % or more of collagen.

15. The method for manufacturing a proteoglycan-containing composition according to claim 14, the composition containing 36 mass % or more of proteoglycans and 36 mass % or more of collagen, and the mass ratio of the proteoglycans and the collagen being 1:1.7 to 1.25:1.

16. The method for manufacturing a proteoglycan-containing composition according to claim 14, wherein the weight-average molecular weight of the proteoglycans is 2,000,000 to 4,150,000 Daltons.

17. The method for manufacturing a proteoglycan-containing composition according to claim 14, wherein the lipid content is 1 mass % or less.

18. The method for manufacturing a proteoglycan-containing composition according to claim 14 wherein proteoglycans having a weight-average molecular weight within the range of 2,000,000 to 3,400,000 Daltons occupy 30 mass % or more of the composition.

19. A method for manufacturing a proteoglycan-containing composition, characterized in comprising: a mincing step for forming a starting material into surimi, where raw cartilage that has not had a history of reaching temperature of 30° C. or higher and has not undergone freezing derived from fish is used as the starting material; and an extraction step for adding an aqueous solvent to the surimi obtained in the mincing step to carry out extraction.

20. The method for manufacturing a proteoglycan-containing composition according to claim 19, wherein the method includes a drying step for furthermore drying extracted articles obtained in the extraction step.
